SEQUENCE LISTING (4 sequences)

SEQ ID NO: 1 - DNA, 744 bp, Oryza sativa
ccacgcgtccgagcaactaggtatctagctaagcagtggtgtgatcagtaggaagttgcaggtgggggatatatcaccaatggctccggtcagcatctccgacgagcgcgccgtctcggtgtcggcggagcgggtgtggaaggtcttctccgacgcgcccgccatgcccaaggtttgtgccggcttcatcgacgccattgaggtcgagggggatggcggggcgggcactgtcaccaccatgaagctcaaccctgctgtggatgatggggggtcattcaaaacacgtgtggtggcacgtgacaacgcagctcacattatcaagtcagaggttctggatgtgccggccggaagtaaagtgggcaagctcaagtcgcacgtgacagagacgaagatcgaggccgccggtgccggctcttgcttggccaagataaacgtggagtatgagctcgaggacggcggctcactgtcgccggagaaggagaagctcatcctcgacggctacttcggcatgctcaagatgatcgaggactacctcgtcgctcaccctaccgagtatgcttaaaaattgtcataaaccaaaataatatacatccatctccgtattgctgcttcctgataattaaataatgtgagccacgacaaatccaatcttttgtggtgtttgattttgtgagagtgatttgtgtttgaggttatgtaagaaataaatcataattgtgatcgtgttctaaaaaaaaaaaaaaa

SEQ ID NO: 2 - protein, 160 aa, Oryza sativa
MAPVSISDERAVSVSAERVWKVFSDAPAMPKVCAGFIDAIEVEGDGGAGTVTTMKLNPAVDDGGSFKTRVVARDNAAHIIKSEVLDVPAGSKVGKLKSHVTETKIEAAGAGSCLAKINVEYELEDGGSLSPEKEKLILDGYFGMLKMIEDYLVAHPTEYA

SEQ ID NO: 3 - DNA, 20 bp, Artificial
  primer No.1
gtgtgatcagtaggaagttg

SEQ ID NO: 4 - DNA, 20 bp, Artificial
  primer No. 2
acctcaaacacaaatcactc

(19) United States
(12) Patent Application Publication
(10) Pub. No.: US 2009/0100552 A1
(43) Pub. Date: Apr. 16, 2009

(54) TRANSGENIC PLANT TRANSFORMED WITH STRESS-RESPONSIVE GENE

(76) Inventors: Tomokazu Koshiba, Tokyo (JP); Teruhiko Terakawa, Kanagawa (JP); Hisakazu Hasegawa, Kanagawa (JP); Setsuko Komatsu, Ibaraki (JP); Takashi Okamoto, Tokyo (JP); Toshiko Furukawa, Tokyo (JP); Kentaroh Shimaya, Kanagawa (JP)

Correspondence Address: FISH & RICHARDSON P.C., P.O BOX 1022, Minneapolis, MN, 55440-1022, US

(21) Appl. No.: 12/162,309

(22) PCT Filed: Jan. 16, 2007
(86) PCT No.: PCT/JP2007/050522

§ 371 Date: Dec. 5, 2008

(30) Foreign Application Priority Data
Jan. 27, 2006 (JP) 2006-18661

(51) Int. Cl. A01H 5/00; C12N 15/82

(52) U.S. Cl. 800/320.2; 435/468

(57) ABSTRACT

A plant having improved heat tolerance and/or increased growth is produced by steps of: introducing DNA into a plant cell to produce a transformed plant cell, wherein the DNA is selected from the group consisting of: (a) DNA coding for a protein consisting of the amino acid sequence represented by SEQ ID NO: 2, (b) DNA including the coding region of the nucleotide sequence represented by SEQ ID NO: 1, (c) DNA coding for a protein having an amino acid sequence represented by SEQ ID NO: 2 in which one or a few amino acids are substituted, deleted, inserted, and/or added, and being involved in growth promoting effect and heat tolerance, (d) DNA hybridizing with DNA consisting of the nucleotide sequence represented by SEQ ID NO: 1 under stringent conditions and coding for a protein which is involved in growth promoting effect and heat tolerance, and (e) DNA coding for a protein having an amino acid sequence having 90% or more identity with the amino acid sequence represented by SEQ ID NO: 2 and being involved in growth promoting effects, heat tolerance, drought tolerance and salt tolerance; and regenerating a transformed plant from the transformed plant cell.

### TECHNICAL FIELD

The present invention relates to a method of producing a plant having enhanced heat tolerance or increased growth.

### BACKGROUND ART

In general, plants existing in nature are exposed to various environmental stresses such as drought stress, high temperature stress, low temperature stress, and salt stress. In particular, global environmental changes are leading to increases in carbon dioxide concentrations and global warming associated therewith, and furthermore, desertification of cultivated lands for crops and the like. In addition, environmental stresses due to drought and high salt concentration are the main environmental causes of the reductions in productivity of agricultural crops. Therefore, it is considered to be an important issue to provide tolerance to various environmental stresses to crops, horticultural plants, and plants for afforestation in terms of reducing labor in cultivation, expansion of cultivatable area, prevention of desert expansion, and afforestation.

In recent years, it has been shown that stress tolerance to salt, drought and the like can be enhanced by introducing environmental stress genes into plants by using genetic engineering techniques, and attempts have been made to produce drought tolerant plants using such techniques.

For example, methods are known to obtain plants having improved salt tolerance, drought tolerance and low temperature stress tolerance by introducing both a choline dehydrogenase gene and a betaine aldehyde dehydrogenase gene (Patent Document 1), a DREB gene (Patent Document 2 and Patent Document 3), a betaine synthesizing enzyme gene (Patent Document 4), a polyamine metabolism related enzyme gene (Patent Document 5), a YK1 gene (Patent Document 6), a gene coding for a glutathione peroxidase-like protein derived from eukaryotic algae (Patent Document 7), an SRK2C gene (Patent Document 8), a polyamine metabolism related genes (Patent Document 9), and the like into plants.

The present inventors previously identified RO-292 as a protein for which the amount significantly increases by salt and drought treatment (Patent Document 10). It was confirmed that RSI1, which is considered to code for the protein, responds to drought stress and salt stress independently of abscisic acid. However, it has not been confirmed that this RSI1 is involved in heat tolerance and growth promotion. Moreover, it has not been confirmed whether a transformed plant into which RSI1 is introduced will have drought tolerance and salt tolerance.

### Patent Document 1: JP08-266179A

### Patent Document 2: JP2000-116260A

### Patent Document 3: JP2000-116259A

### Patent Document 4: JP2003-143988A

### Patent Document 5: JP2004-180588A

### Patent Document 6: JP2004-261136A

### Patent Document 7: JP2005-073505A

### Patent Document 8: JP2005-253395A

### Patent Document 9: JP2005-237387A

### Patent Document 10: JP2003-334084A

### DISCLOSURE OF THE INVENTION

### Problems to be Solved by the Invention

There is a demand for plants that can grow under high temperatures and strong insolation in low latitude areas and the like, in addition to being able to tolerate the above-described environmental stresses such as salt tolerance and drought tolerance. A direct link between the ability to grow under high temperatures and strong insolation, that is, heat tolerance, salt tolerance, and drought tolerance has yet to be found.

In recent years, particularly with regard to agricultural crops, attempts have been made to increase yields, shorten growing periods, and raise the efficiency of food production. Moreover, shorten growing period is also a characteristic that is desired for horticultural plants, grasses and the like. In particular, it would be extremely significant to produce plants that have tolerance to high temperatures and strong insolation, and that can grow rapidly in low latitude areas.

Therefore, it is an object of the present invention to produce a plant having increased growth and a plant having enhanced heat tolerance.

### Means for Solving the Problems

The present inventors have conducted intensive research in order to solve the above-described problems, and they have succeeded in providing a transformed plant having increased growth and/or enhanced heat tolerance by introducing the RSI1 (Patent Document 10 as a reference) gene into a plant.

More specifically, the present invention provides a method for producing a plant having increased growth, including the steps of: introducing DNA into a plant cell to produce a transformed plant cell, wherein the DNA is selected from the group consisting of: (a) DNA coding for a protein consisting of the amino acid sequence represented by SEQ ID NO: 2; (b) DNA including the coding region of the nucleotide sequence represented by SEQ ID NO: 1; (c) DNA coding for a protein having an amino acid sequence represented by SEQ ID NO: 2 in which one or a few amino acids are substituted, deleted, inserted, and/or added, and having a growth promoting effect; (d) DNA hybridizing with DNA consisting of the nucleotide sequence represented by SEQ ID NO: 1 under stringent conditions and coding for a protein which has a growth promoting effect; and (e) DNA coding for a protein having an amino acid sequence having 90% or more identity with the amino acid sequence represented by SEQ ID NO: 2 and having a growth promoting effect; and regenerating a transformed plant from the transformed plant cell.

Moreover, the present invention provides a method of producing a plant having improved heat tolerance, including the steps of: introducing DNA into a plant cell to produce a transformed plant cell, wherein the DNA is selected from the group consisting of: (a) DNA coding for a protein consisting of the amino acid sequence represented by SEQ ID NO: 2; (b) DNA including the coding region of the nucleotide sequence represented by SEQ ID NO: 1; (c) DNA coding for a protein having an amino acid sequence represented by SEQ ID NO: 2 in which one or a few amino acids are substituted, deleted, inserted, and/or added, and being involved in heat tolerance; (d) DNA hybridizing with DNA consisting of the nucleotide sequence represented by SEQ ID NO: 1 under stringent conditions and coding for a protein that is involved in heat tolerance; and (e) DNA coding for a protein having an amino acid sequence having 90% or more identity with the amino acid sequence represented by SEQ ID NO: 2 and being involved in heat tolerance; and regenerating a transformed plant from the transformed plant cell.

In addition, the present invention provides a method of producing a plant having improved heat tolerance and increased growth, including the steps of: introducing DNA into a plant cell to produce a transformed plant cell, wherein the DNA is selected from the group consisting of: (a) DNA coding for a protein consisting of the amino acid sequence represented by SEQ ID NO: 2; (b) DNA including the coding region of the nucleotide sequence represented by SEQ ID NO: 1; (c) DNA coding for a protein having an amino acid sequence represented by SEQ ID NO: 2 in which one or a few amino acids are substituted, deleted, inserted, and/or added, and being involved in growth promoting effects and heat tolerance; (d) DNA hybridizing with DNA consisting of the nucleotide sequence represented by SEQ ID NO: 1 under stringent conditions and coding for a protein which is involved in growth promoting effect and heat tolerance; and (e) DNA coding for a protein having an amino acid sequence having 90% or more identity with the amino acid sequence represented by SEQ ID NO: 2 and being involved in growth promoting effect; and regenerating a transformed plant from the transformed plant cell.

Furthermore, the present invention provides a method of producing a plant having drought tolerance, including steps of: introducing DNA into a plant cell to produce a transformed plant cell, wherein the DNA is selected from the group consisting of: (a) DNA coding for a protein consisting of the amino acid sequence represented by SEQ ID NO: 2; (b) DNA including the coding region of the nucleotide sequence represented by SEQ ID NO: 1; (c) DNA coding for a protein having an amino acid sequence represented by SEQ ID NO: 2 in which one or a few amino acids are substituted, deleted, inserted, and/or added, and being involved in drought tolerance; (d) DNA hybridizing with DNA consisting of the nucleotide sequence represented by SEQ ID NO: 1 under stringent conditions and coding for a protein which is involved in drought tolerance; and (e) DNA coding for a protein having an amino acid sequence having 90% or more identity with the amino acid sequence represented by SEQ ID NO: 2 and being involved in drought tolerance; and regenerating a transformed plant from the transformed plant cell.

In addition, the present invention provides a method of producing a plant having salt tolerance, including steps of: introducing DNA into a plant cell to produce a transformed plant cell, wherein the DNA is selected from the group consisting of: (a) DNA coding for a protein consisting of the amino acid sequence represented by SEQ ID NO: 2; (b) DNA including the coding region of the nucleotide sequence represented by SEQ ID NO: 1; (c) DNA coding for a protein having an amino acid sequence represented by SEQ ID NO: 2 in which one or a few amino acids are substituted, deleted, inserted, and/or added, and being involved in salt tolerance; (d) DNA hybridizing with DNA consisting of the nucleotide sequence represented by SEQ ID NO: 1 under stringent conditions and coding for a protein which is involved in salt tolerance; and (e) DNA coding for a protein having an amino acid sequence having 90% or more identity with the amino acid sequence represented by SEQ ID NO: 2 and being involved in salt tolerance; and regenerating a transformed plant from the transformed plant cell.

The method of the present invention may further include a step of allowing the plant to undergo sexual reproduction or asexual reproduction, thereby producing a progeny plant.

A monocotyledonous plant, particularly one in the family Poaceae, can be used as the plant in the method of the present invention. Also, the present invention relates to a plant obtained by the above-described method.

The present invention can provide a plant which exhibits heat tolerance and having increased growth by obtaining a transformed plant by introducing the RSI1 gene into a plant. The obtained plant can grow under high temperature and under strong light conditions, compared with a plant not having the introduced gene. Moreover, the plant height and leaf thereof become larger and the growth thereof is promoted, compared with a plant not having the introduced gene. The plant can be used in the cultivation of crops in low latitude areas and under strong light, improvement of horticultural plants, cultivar improvement of agricultural crops and the like.

### BEST MODE FOR CARRYING OUT THE INVENTION

The present invention will be described in detail hereinafter. The transformed Poaceae plant according to the present invention having the introduced stress responsive gene can be produced by the following method.

Construction of Recombinant DNA Including a Stress Response Gene

In the present invention, a plant having increased growth and/or improved heat tolerance can be obtained by introducing DNA coding for the RSI1 protein into a plant cell, and regenerating the plant cell into a plant or a whole plant.

DNA coding for the RSI1 protein (which will be referred to as the RSI1 gene hereinafter) specifically includes DNA described in root-specific gene RSOsPR10, which responds to stress derived from rice [Plant Cell Physiology Vol. 45, Issue 5, pp. 550-559, 2004; JP2003-334084A].

The RSI1 gene to be used in the present invention was identified by the present inventors as a gene coding for a protein for which production is induced by treatment using sodium chloride or by drought, but is not induced by abscisic acid in the root of a plant. More specifically, the RSI1 gene is a gene which was obtained by treating a plant with a stress treatment such as exposure to salt or drought, isolating proteins specifically expressed as a result of the treatment from the treated leaf or root, and analyzing the gene sequences. The present invention was accomplished by further revealing that the RSI1 gene is a gene which provides heat tolerance and a growth promoting effect. In general, proteins which are expressed in response to these various environmental changes (stresses) include PR (pathogenesis-related) proteins, and they are considered to be one of the plant biological defense proteins against environmental stresses.

The RSI1 protein may include not only DNA coding for the so-called “native form” of the protein (SEQ ID NO: 1), but also mutant proteins having an amino acid sequence of the RSI1 protein (SEQ ID NO: 2) in which one or a few amino acids are substituted, deleted, inserted, and/or added, and having a function to provide heat tolerance and/or a growth promoting effect to a plant. In addition, it may also include mutant proteins having an amino acid sequence having 90% or more identity with the amino acid sequence represented by SEQ ID NO: 2 and having a function to provide heat tolerance and/or a growth promoting effect to a plant.

The RSI1 protein is a protein in which the sequence was determined by the present inventors and is represented by SEQ ID NO: 2. DNA coding for the RSI1 protein derived from rice is represented by SEQ ID NO: 1.

DNA selected from the group consisting of (a) DNA coding for a protein consisting of the amino acid sequence represented by SEQ ID NO: 2, (b) DNA including the coding region of the nucleotide sequence represented by SEQ ID NO: 1, (c) DNA coding for a protein having an amino acid sequence represented by SEQ ID NO: 2 in which one or a few amino acids are substituted, deleted, inserted, and/or added, (d) DNA hybridizing with DNA consisting of the nucleotide sequence represented by SEQ ID NO: 1 under stringent conditions, and (e) DNA having 90% or more identity in amino acid sequence with the amino acid sequence represented by SEQ ID NO: 2 can be used as DNA coding for the RSI1 protein. The DNAs in the above (a) through (e) are DNAs coding for proteins involved in growth promoting effect, heat tolerance, drought tolerance and/or salt tolerance.

DNA coding for the RSI1 protein of the present invention includes genomic DNA, cDNA, and chemically synthesized DNA. The preparation of genomic DNA and cDNA can be performed by a common method for one skilled in the art. For example, genomic DNA can be prepared by extracting genomic DNA from a rice cultivar having the RSI1 gene, preparing a genomic library (plasmid, phage, cosmid, BAC, PAC and the like can be used as a vector), developing the same, and performing colony hybridization or plaque hybridization by using a probe prepared based on DNA coding for the protein of the present invention (for example, SEQ ID NO: 1). In addition, it can be prepared by preparing a primer specific for DNA coding for the protein of the present invention (for example, SEQ ID NO: 1), and performing PCR by using the same. Moreover, for example, cDNA can be prepared by synthesizing cDNA based on mRNA extracted from a rice cultivar having the RSI1 gene, inserting the same into a vector such as λZAP to prepare a cDNA library, developing the same, and performing colony hybridization or plaque hybridization by the same method as the above-described method, or performing PCR.

Mutants, derivatives, alleles, homologs, or orthologs coding for proteins having an amino acid sequence represented by SEQ ID NO: 2 in which one or a few amino acids are substituted, deleted, inserted, and/or added can be used. Mutations can also be artificially introduced. Mutation in a nucleotide sequence resulting in a mutation in the amino acid sequence of the encoded protein can occur in nature. An example of commonly known methods for one skilled in the art of preparing DNA coding for a protein for which the amino acid sequence is altered includes the site-directed mutagenesis method (Kramer, W. & Fritz, H.-J. (1987) Oligonucleotide-directed construction of mutagenesis via gapped duplex DNA. Methods in Enzymology, 154: 350-367). As described above, DNA of the present invention even includes DNA coding for a protein having an amino acid sequence coding for the native form of the RSI1 protein in which one or a few amino acids are substituted, deleted or added, as long as it encodes a protein having a function equivalent to that of the native form of the RSI1 protein (SEQ ID NO: 2). Moreover, there are cases in which even though the nucleotide sequence is mutated, the mutation is not associated with a mutation in the amino acids of the protein (degeneration mutation). DNA of the present invention even includes such condensation mutants.

Other examples of commonly known methods for one skilled in the art of preparing DNA coding for a protein which is functionally equivalent to the RSI1 protein represented by SEQ ID NO: 2 include methods utilizing hybridization techniques (Southern, E. M. (1975) Journal of Molecular Biology, 98, 503) and polymerase chain reaction (PCR) techniques (Saiki, R. K. et al. (1985) Science, 230, 1350-1354, Saiki, R. K. et al. (1988) Science, 239, 487-491). More specifically, it is something that one skilled in the art can usually perform to isolate DNA having a high homology with the RSI1 gene from rice and other plants by using the nucleotide sequence of the RSI1 gene (SEQ ID NO: 1) or a part thereof as the probe and an oligonucleotide which specifically hybridizes with the RSI1 gene as the primer. As described above, DNA coding for a protein having a function equivalent to the RSI1 protein which can be isolated by the hybridization technique and PCR technique is also included in the DNA of the present invention.

To isolate such DNA, a hybridization reaction is preferably performed under stringent conditions. The term “stringent hybridization conditions” in the present invention refers to the conditions of 6 M urea, 0.4% SDS and 0.5×SSC, or hybridization conditions of equivalent stringency. However, the conditions are not limited to these conditions. Conditions of even higher stringency such as 6 M urea, 0.4% SDS, and 0.1×SSC can be expected to isolate DNAs of higher homologies. On the other hand, one may perform hybridization under lower stringency conditions such as lower temperature conditions, and hybridization by increasing salt concentration as long as it has a function equivalent to the RSI1 protein.

A plurality of factors such as temperature and salt concentration are considered to be the factors which affect hybridization stringency. One skilled in the art can establish an optimal stringency by appropriately selecting these factors. DNA isolated by the above-described method at the amino acid level is considered to have a high homology with the amino acid sequence of the RSI1 protein (SEQ ID NO: 2). The term “high homology” refers to identities of at least 50% or more, more preferably 70% or more, and even more preferably 90% or more (for example, 95% or more) over the entire amino acid sequence.

Amino acid sequence identities and nucleotide sequences identities can be determined by using the BLAST algorithm by Karlin and Altschul (Karlin, S., Altschul, S. F., Proc. Natl. Acad. Sci. USA, 87: 2264-2268, 1990; Karlin, S., Altschul, S. F., Proc. Natl. Acad. Sci. USA, 90: 5873-5877, 1993). BLASTN and BLASTX programs have been developed based on the BLAST algorithm (Altschul, S. F. et al., J. Mol. Biol., 215: 403, 1990). When nucleotide sequences are analyzed by using BLASTN, parameters are set to be, for example, score=100 and wordlength=12. In addition, when amino acid sequences are analyzed by using BLASTX, parameters are set to be, for example, score=50 and wordlength=3. When BLAST and Gapped BLAST programs are used, default parameters for the respective program are used. Specific procedures of these analysis methods are publicly known (http://www.ncbi.nlm.nih.gov/).

In order to introduce DNA into a plant cell, it is preferred to ligate DNAs in the above (a) through (e) to a vector for insertion. For example, DNA in the above (a) through (e) can be ligated into a vector DNA by ligating a promoter upstream thereof and a terminator downstream thereof in order to allow then to constantly function in a rice cell. It is easy for one skilled in the art to optimize such promoters and the like.

Either constitutive promoters or inductive promoters can be used as the promoter to be used in the present invention. In addition, promoters specific for tissues or organs such as the root and leaf can be also used. Examples of promoters include the CaMV35S promoter derived from Cauliflower Mosaic Virus [The EMBO J., Vol. 6, p. 3901, 1987], and the ubiquitin promoter derived from corn [Plant Mol. Biol., Vol. 23, p. 567, 1993]. Examples of the terminator include the terminator derived from Cauliflower Mosaic Virus and the terminator derived from the nopaline synthase gene. However, they are not limited to the above as long as they are promoters and terminators which function in the plant.

In order to facilitate the selection of rice having a introduced vector including a stress response gene, a selection marker gene can be simultaneously used and introduced in combination with the stress response gene. As the selection marker gene to be used in such cases, one or more genes selected from the hygromycin phosphotransferase gene (HPT) that is resistant to the antibiotic hygromycin, phosphinothricin acetyltransferase that is resistant to the herbicide phosphinothricin, neomycin phosphotransferase gene that is resistant to kanamycin and gentamicin, and the like can be used. However, this is not limited to these as long as the gene is a gene which functions as a selection marker. Preferably, the hygromycin phosphotransferase gene is used.

Gene Introduction into a Plant in the Poaceae and Preparation of a Transformed Plant

The present invention can be applied to any plant, and more specifically, monocotyledonous plants and dicotyledonous plants, preferably used to insert into to monocotyledonous plants, and even more preferably to plants in the Poaceae. Examples of a plant in the Poaceae include rice, corn, wheat, barley, rye, oat, lawngrass (bentgrass, Zoysia japonica, Zoysia matrella), and fescues (tall fescue and red fescue). Examples of a plant in the Poaceae to be used for gene introduction include tissues of various organs such as the seed, root, stem, and leaf, and further include a plant cell such as in a callus and a protoplast. The callus is preferred.

Subsequently, the gene introduction into a plant in the Poaceae and preparation of a transformed plant is performed. A recombinant DNA including a stress response gene can be introduced by a publicly known gene introduction method. Examples of a publicly known gene introduction method include the electroporation method, polyethylene glycol method, agrobacterium method, and particle gun method (Experimental Protocol of Model Plants, Rice and Shovelwood, Cell Engineering supplement: Plant Cell Engineering Series, Vol. 4, Shujun Sha, pp. 89-98, Shimamoto et al., 1996), Whisker method (Japanese Patent No. 3312867B), or methods conforming thereto. These methods are publicly known to one skilled in the art. Preferred gene introduction method to be used in the present invention is the agrobacterium method.

Tissue fragments on which gene introduction is to be performed are placed on a selection medium prepared by the following method. The selection medium is a solid medium which is solidified by adding a specific gelatinizing agent after adding specific plant hormones, oxygen sources, and vitamins to a specific base medium. For example, it is prepared by adding a range of 10 to 60 g/l, preferably 20 to 40 g/l of sucrose, a range of 0.01 to 10 mg/l, preferably 0.1 to 5 mg/l of auxins, and a range of 0.01 to 10 mg/l, preferably 0.1 to 5 mg/l of cytokinins as plant hormones, and a range of 1 to 50 mg/l, preferably 10 to 30 mg/l of acetosyringone, respectively, to MS medium in which inorganic salt concentration is maintained as it is or is diluted to 1/2 to 1/3 concentration, adjusting the pH in the range of 4 to 7, preferably 5 to 6, and adding a range of 1 to 10 g/l, preferably 2 to 4 g/l, of gellan gum.

In the selection step of the present invention described above, examples of the plant hormones to be added to the medium include 2,4-D, naphthaleneacetic acid (NAA), indoleacetic acid (IAA) and the like as auxins, and benzyladenine (BA), kinetin, thidiazuron, and the like as cytokinins.

A selection agent is added to the selection medium in accordance with the type of the selection marker gene in order to efficiently select the target transformed cell. For example, hygromycin is added at a concentration in the range of 10 to 100 mg/l, preferably 30 to 50 mg/l, as a selection agent.

Furthermore, as a sterilizing agent for agrobacterium bacteria in which gene introduction treatment has been completed, carbenicillin is added at a concentration in a range of 50 to 500 mg/l, and preferably 100 to 300 mg/l. This is cultured in a range of 10 to 30° C., preferably 25 to 30° C., in the dark, for 20 to 90 days, preferably 30 to 60 days, to select a callus having the introduced recombinant DNA.

The selected callus cultured by the above-described method is placed on a growth medium prepared by the following method to grow a plant. The plant growth medium is a solid medium which is solidified by adding a specific gelatinizing agent after adding specific plant hormones, oxygen sources and vitamins to a specific base medium. For example, it is prepared by adding a range of 10 to 60 g/l, preferably 20 to 40 g/l of sucrose, a range of 0.01 to 10 mg/l, preferably 0.1 to 5 mg/l of auxins, a range of 0.01 to 10 mg/l, preferably 0.1 to 5 mg/l of cytokinins as plant hormones, and a range of 50 to 1000 mg/l, preferably 300 to 500 mg/l of carbenicillin, respectively, to an MS medium in which the inorganic salt concentration is maintained as it is or is diluted to 1/2 to 1/3 concentration, adjusting the pH in the range of 4 to 7, preferably 5 to 6, and adding a range of 1 to 10 g/l, preferably 2 to 4 g/l, of gellan gum.

In the regeneration step of the present invention described above, examples of the plant hormones to be added to the medium include 2,4-D, naphthaleneacetic acid (NAA), indoleacetic acid (IAA) and the like as auxins, and benzyladenine (BA), kinetin, thidiazuron and the like as cytokinins.

This is cultured at a range of 10 to 30° C., preferably 25 to 30° C., in the light for 30 to 120 days, preferably 30 to 60 days, to regenerate and grow a transformed plant from the callus. The term “in the light” refers to an illuminance in the range of 500 to 10000 lux, preferably 500 to 2000 lux, for 5 to 24 hours, preferably 14 to 18 hours of duration of light exposure per day.

Regeneration of a plant from the transformed plant cell can be performed by a method publicly known to one skilled in the art in accordance with the type of plant cell (Toki et al. (1992) Plant Physiol. 100: 1503-1507). For example, several techniques have been already established and are widely used in the technical field of the present invention as a method of making a transformed complete rice. Examples include the method which allows regeneration of a plant after gene introduction into a protoplast by polyethylene glycol (suitable for Indian rice cultivars) (Datta, S. K. (1995) In Gene Transfer to Plants (Potrykus I and Spangenberg Eds.) pp. 66-74), the method which allows regeneration of a plant after gene introduction into a protoplast by electric pulse (Toki et al. (1992) Plant Physiol. 100: 1503-1507), the method which allows regeneration of a plant after directly introducing a gene into the cell by the particle gun method (Christou et al. (1991) Bio/technology, 9: 957-962), and the method which allows regeneration of a plant after introducing a gene via agrobacterium (the method for extremely rapid transformation of a monocotyledonous plant (Japanese Patent No. 3141084B)). These methods can be preferably used in the present invention.

Confirmation of Introduction of Gene into Plant

In order to confirm the introduction of a target stress response gene into the transformed cell (callus) and transformed plant obtained by the above-described step, DNA is extracted from these cells in accordance with the known protocol of the art, followed by performing the publicly known PCR (polymerase chain reaction) method or Southern hybridization method.

Confirmation of Heat Tolerance and Growth of Transformed Plant

Growth promotion and tolerance of high temperature and/or strong light of the transformed plant having the introduced RSI1 gene can be confirmed by an easy detection method.

The term “growth or development is promoted” in this specification refers to the fact that the leaf size, plant height, stem size, leaf weight, root length, and the like are larger, compared with those of an untreated plant of the same species. Alternatively, it refers to the fact that the leaf size, plant height, stem size, leaf weight, root length, and the like can grow larger, compared with those of an untreated plant of the same species, in the same period of time.

The term “heat tolerance” in this specification refers to tolerance of strong light and/or high temperature stress. The term “strong light stress” refers to stress which a plant experiences when placed in an environment in which the amount of light exceeds that suitable for the growth of the plant, and the fact that the physiological functions of the plant tissues and cells are impaired and damaged due to the strong light. The term “high temperature stress” refers to the stress which a plant experiences when placed in an environment in which the temperature exceeds that suitable for the growth of the plant, and the fact that the physiological functions of the plant tissues and cells are impaired and damaged due to the high temperature. The term “enhanced heat tolerance” refers to the fact that a plant can grow under strong light and/or under high temperature, compared with an untreated plant of the same species.

Furthermore, a growth evaluation test of the transformed Poaceae plant having the introduced RSI1 gene can be confirmed by cultivating the same in a range of 20 to 30° C.

Once a transformed whole plant in which the DNA of the present invention is introduced in the genome, is obtained, a progeny plant can be obtained by sexual reproduction or asexual reproduction of the plant. In addition, it is also possible to obtain propagation materials (for example, seeds, fruits, grafts, tubers, tuberous roots, roots, calluses and protoplasts) from the plant as well as progeny or clones thereof, and to produce the plants therefrom in large quantities. The present invention includes a plant cell having introduced DNA of the present invention, a plant including the cell, a progeny and clone of the plant, and a propagation material from the plant as well as progeny and clones thereof. The growth of plant produced by the above-described method is considered to be promoted, and/or heat tolerance thereof is enhanced, compared with the wild-type plant. By using the method of the present invention, productivity of useful agricultural crops, such as rice, can be enhanced, and this is highly valuable.

Examples of an embodiment of the present invention include the following.

(1) A transformed plant having the introduced stress response gene.

(2) The transformed plant according to the above (1), wherein the stress response gene is a gene coding for a protein derived from rice.

(3) The transformed plant according to the above (1) or (2), wherein the plant is a plant in the Poaceae.

(4) The transformed plant according to the above (1), characterized in that the plant is a plant having improved drought tolerance.

(5) The transformed plant according to the above (1), characterized in that the plant is a plant having improved salt tolerance.

(6) The transformed plant according to the above (1), characterized in that the plant is a plant having improved heat tolerance.

(7) The transformed plant according to the above (1), characterized in that the growth of the transformed plant is promoted.

### EXAMPLES

The present invention will be explained specifically by exemplifying examples hereinafter. However, the scope of the present invention is not limited by these examples. Unless otherwise specified, the following experimental procedure was in accordance with the method described in Molecular Cloning 2^{nd} Edition (J. Sambrook et al., Cold Spring Harbor Laboratory Press, 1989).

### Example 1

### Preparation of Transformed Rice with the RSI1 Gene

(1) Construction of Recombinant DNA Including RSI1 Gene Derived From Rice

An expression vector was prepared in order to constantly express RSI1 gene in a plant by using the stress response gene (RSI1) derived from rice as a gene to be introduced into a plant.

Restriction enzyme sites BamHI were added to the 5′ side and 3′ side of the above-described stress response gene RSI1 DNA so that they included an open reading frame by the PCR method. RSI1, wherein BamHI sites were added to the 5′ end and 3′ end thereof, was inserted to the pBluescriptIISK-vector. This was treated with the restriction enzyme BamHI to cut out the RSI1 gene. The ends of the fragment were then made blunt by using a DNA Blunting Kit (Takara Bio Inc.), and the fragment was purified by the glass milk method. On the other hand, the publicly known expression vector for rice having Cauliflower Mosaic Virus 35S promoter, pIG121-Hm (obtained from Tokyo Metropolitan University) was cleaved with the restriction enzymes XbaI and SacI, and the ends were made blunt by using a DNA Blunting Kit (Takara Bio Inc.). The above-described RSI1 gene was ligated in the downstream of the 35S promoter within the pIG121-Hm vector by the DNA Ligation Kit method (Takara Bio Inc.). The plasmid vector was named pBIH1-IG.

Agrobacterium tumefaciens EHA101/pBIH1-IG was prepared by introducing the recombinant vector pBIH1-IG toto agrobacterium tumefaciens (EHA101) by the publicly known freezing and thawing method (for example, Cell Engineering, 1992, Vol. 4, Issue 3, pp. 193-203).

The recombinant vector pBIH1-IG has the stress response gene (RSI1) linked between the 35S promoter derived from the Cauliflower Mosaic Virus and the NOS terminator derived from nopaline synthase, hygromycin phosphotransferase gene (HPT) linked between the 35S promoter and the terminator derived from agrobacterium, and neomycin phosphotransferase gene (NPT) linked between the nopaline synthase promoter (Pnos) derived from agrobacterium and the terminator.

(2) Gene Introduction into Rice Tissues

Mature seeds of rice (cultivar: Nipponbare) were immersed in a sodium hypochlorite solution, having an effective concentration of chlorine 1%, for 60 minutes, for sterilization. One hundred seeds per experimental section were placed on a solid medium which was prepared by adding 3% sucrose and 2 mg/l of 2,4-D as a plant hormone to N6 medium, adjusting the pH to 5.8, and adding 0.3% gel lyte. The container to be used for this culture was a sterilized plastic petri dish (9 cm in diameter, 1.5 cm in height), and 20 tissue fragments were placed in each petri dish. This was cultured at 28° C. in the dark for 30 days to induce calluses.

One hundred calluses of the above-described rice were subjected to immersion treatment for 2 minutes in a culture medium, in which agrobacterium tumefaciens (EHA101/pBIH1-IG) including the recombinant vector pBIH1-IG obtained by the above-described method was cultured overnight at 28° C. in LB medium. After the immersion treatment, the calluses were placed on a solid medium which was prepared by adding 3% sucrose, and 2 mg/l of 2,4-D and 10 mg/l of acetosyringone as plant hormones, respectively, to N6 medium, adjusting the pH to 5.8, and adding 0.3% gel lyte. This was co-cultured under conditions of 25° C. in the dark for 3 days.

One hundred calluses after the co-culture were transferred to a sterilized 50 ml centrifuge tube (27 mm in inner diameter, 115 mm in length), and rinsed by adding 30 ml of a rinse solution (which was prepared by adding 300 mg of carbenicillin to MS liquid medium with 1/2 inorganic salt concentration). Furthermore, after repeating this procedure twice, excess water was removed by using a sterilized filter paper. The rinsed tissue fragments were placed on a solid selection medium which was prepared by adding 3% sucrose, 2 mg/l of 2,4-D as a plant hormone, 500 mg/l of carbenicillin and 50 mg/l of hygromycin, respectively, to N6 medium, adjusting the pH to 5.8, and adding 0.3% gel lyte. This was transplanted to fresh selection medium every month under conditions of 28° C. in the dark.

(3) Regeneration of Transformed Rice Plant

After being transplanted to the selection medium, hygromycin resistant transformed calluses were formed. These calluses were placed on a solid regeneration medium which was prepared by adding 3% sucrose, 1 mg/l of NAA and 2 mg/l of BA as plant hormones, 300 mg/l of carbenicillin and 50 mg/l of hygromycin, respectively, to MS medium, adjusting the pH to 5.8, and adding 0.3% gel lyte. These were cultured under conditions of 28° C. in the light (1000 lux, for 16 hours of light). As a result, 19 rice whole plants transformed by the stress response gene RSI1 were obtained.

These plants were all subjected to acclimation and naturalization after being allowed to undergo rhizogenesis sufficiently. These transformed plant bodies were cultivated in an incubator under conditions of 28° C. in the light (20000 lux, for 16 hours of light). Subsequently, the transformed plants (T1 generation) were cultivated in a closed greenhouse to harvest the seeds (T2 generation). These progeny seeds were subjected to a resistance test (segregation ratio test) on a medium including the antibiotics hygromycin as a selection marker to obtain homozygous strains (T3 generation and T4 generation).

(4) Analysis of Introduced Gene

The leaves of the transformed plants obtained in the above (3) were used as materials for gene analysis. The whole DNAs were extracted from 10 mg of the rice leaf discs in accordance with the publicly known CTAB method. These were checked for the introduced gene by the PCR method. The following primers prepared by chemical synthesis were used in combination as the primers.

(a) Primer No. 1 (represented by SEQ ID NO: 3)
5′-GTGTGATCAGTAGGAAGTTG-3′
(b) Primer No. 2 (represented by SEQ ID NO: 4)
5′-ACCTCAAACACAAATCACTC-3′

One μl of DNA solution of each of the transformed rice strains extracted by the above-described method was used as the template. The amplification reaction solution to be used in the reaction was prepared using a PCR kit (Takara Shuzo Co., Ltd., LA PCR Kit Ver 2.1).

The above-described amplification reaction of DNAs by the PCR method was performed by repeating 35 cycles of 3 reaction procedures, each cycle consisting of denaturation at 94° C. for 30 seconds, annealing at 55° C. for 1 minute and extension at 72° C. for 30 seconds, by using the PCR reaction machine (ASTEK Co., Ltd., Program Temp Control System PC-700).

After PCR, a part of the reaction solution was subjected to electrophoresis in a 1.2% agarose gel to compare the presence and size of the bands. As a result, transformation with the RSI1 gene was confirmed in all the plants.

(5) Examination of Environmental Stress Tolerance of Transformed Rice

The seeds of a homozygous fixed strain of the transformed rice (T4 generation) and common cultivar (Nipponbare) obtained in the above (3) were used for the following performance evaluations.

### A) Evaluation of Salt Tolerance

Ten seeds of each strain of the homozygous strain of the transformed rice (Strain No: S3) and the non-transformed rice Nipponbare cultivar were allowed to germinate in tap water. Subsequently, HYPONeX at 1/1000 concentration, including a predetermined concentration of sodium chloride (50 mM) was added thereto, and the plants were grown in an incubator under conditions of 27° C. for a 12-hour period of light and a 12-hour period of darkness for 14 days. Subsequently, after 14 days, the plant height and root length of each seedling were measured. The results are shown in Table 1.

TABLE 1

| | Plant height | Root length |
|---|---|---|
| Strain No. | (mm) | (mm) |
| Transformed rice S3 | 22 | 15 |
| Non-transformed rice Nipponbare | 13 | 4 |

The results in Table 1 show that both the plant height and root elongation of the non-transformed rice were significantly suppressed in the 50 mM NaCl solution. In comparison, the plant height and root length of the S3 strain of the transformed rice were obviously greater, indicating that tolerance to salt is enhanced.

### (B) Evaluation of Drought Tolerance

Four seeds of each strain of the homozygous strain of the transformed rice (Strain No: S3) and the non-transformed rice Nipponbare cultivar were placed in test tubes filled with tap water at one seed per tube. They were cultivated in an incubator under temperature conditions of 28° C. in the light (20000 lux, for 12 hours of light) for 14 days. Subsequently, each seedling was left to stand on a clean bench for 5 hours for air drought treatment, and was then cultivated again under the same conditions as above by returning it to a growth medium. After 7 days, the number of growing stalks and dead stalks of each seedling were examined. The results are shown in Table 2.

TABLE 2

| | Number of | Number of |
|---|---|---|
| Strain No. | growing stalks | dead stalks |
| Transformed rice S3 | 4 | 0 |
| Non-transformed rice Nipponbare | 1 | 3 |

The results in Table 2 show that most of the non-transformed rice died by air drought treatment. In comparison, the S3 strain of the transformed rice grew normally, indicating that tolerance to drought is enhanced.

(6) Examination of the Growth of the Transformed Rice

Five seeds of each strain of the homozygous strains of the transformed rice (Strains No: S2, S3, S4 and S19) and Nipponbare cultivar were cultivated in a Wagner pot filled with granular soil (marketed as the product name “Kumiai granular soil”), and cultivated under conditions in a closed greenhouse. After cultivation initiation, the cultivation was completed when the uppermost leaf developed, and the plant heights were measured. Furthermore, the twelfth leaf was cut to measure the leaf weights. The results are shown in Table 3.

TABLE 3

| Strain No. | Plant height | Leaf weight |
|---|---|---|
| Transformed rice S2 | 92 | 0.6 |
| Transformed rice S3 | 105 | 0.7 |
| Transformed rice S4 | 82 | 0.4 |
| Transformed rice S19 | 90 | 0.5 |
| Non-transformed rice, Nipponbare | 70 | 0.4 |

Compared with those of the non-transformed rice, the plant heights and leaf weights of strains S2, S3, S4 and S19 of the transformed rice were significantly larger, indicating that their growths were promoted.

### Example 2

### Preparation of Bentgrass Transformed with RSI1 Gene

(7) Construction of Recombinant DNA Including Stress Response Gene Derived from Rice for Bentgrass Gene Introduction

An expression vector was prepared in order to constantly express RSI1 gene in a bentgrass plant. The above-described stress response gene RSI1 was inserted into the pBluescriptIISK-vector after restriction enzyme sites BamHI were added to the 5′ side and 3′ side of DNA so that they included an open reading frame based on the nucleotide sequence by the PCR method. This was treated with the restriction enzyme BamHI to cut out the RSI1 gene. Subsequently, the ends of the fragment were then made blunt by using a DNA Blunting Kit (Takara Bio Inc.), and the fragment was purified by the glass milk method. On the other hand, the publicly known plant cell transformation vector pBI221 having the Cauliflower Mosaic Virus 35S promoter (Clontech Laboratories, Inc.) was cleaved with the restriction enzymes XbaI and SacI, and the ends were made blunt by using a DNA Blunting Kit (Takara Bio Inc.). The above-described RSI1 gene was ligated to the downstream of 35S promoter within pBI221 vector by the DNA Ligation Kit method (Takara Bio Inc.). The plasmid vector was named pBIH2.

(8) Introduction of the Recombinant Vector into Bentgrass Callus Cell

Introduction of the recombinant vector pBIH2 obtained by the above-described method into a bentgrass callus cell was performed (Japanese Patent No. 3312867B as a reference).

First, the chaff was removed from the ripe seeds of bentgrass (cultivar: Penncross). The obtained seeds were immersed in a 70% ethanol solution for 1 minute and, subsequently, in a 1% sodium hypochlorite solution (effective concentration of chlorine) for 60 minutes for sterilization treatment of the seeds. The bentgrass seeds sterilized by the above-described method were placed on a solid medium which was prepared by adding 30 g/l sucrose, 500 mg/l of casamino acid, and 6.6 mg/l of dicamba, and 0.5 mg/l of benzyladenine as plant hormones, respectively, to the inorganic component compositions of a publicly known MS medium, adjusting the pH to 5.8, and adding 3 g/l of gel lyte. The container to be used for this culture was a sterilized plastic petri dish (9 cm in diameter, 1.5 cm in height), and 25 seeds were placed in each petri dish. This was cultured at 28° C. in the dark for 60 days to induce calluses. After calluses are formed, these calluses were sifted by using a stainless steal screen sieve with 1 mm pores to obtain 3 ml in PCV (packed cell volume) of calluses having a size of 1 mm or less.

Five mg of whisker LS20 made of potassium titanate were put into a 1.5 ml tube, and this was left to stand for 1 hour. By removing and completely evaporating ethanol, sterilized whisker was obtained. One ml of sterile water was added to a tube containing the whisker, and this was thoroughly agitated. The whisker and sterile water were centrifuged, and the supernatant water was discarded. This rinsing procedure of the whisker was performed 3 times. Subsequently, 0.5 ml of MS liquid medium prepared by adding 30 g/l of sucrose to publicly known MS medium and adjusting the pH to 5.8 was added to the same tube to obtain a whisker suspension.

Two hundred fifty μl of bentgrass calluses having a size of 1 mm or less was added to a tube containing the whisker suspension obtained by the above-described method, and the mixture was agitated. The mixture of the calluses and whisker suspension was centrifuged at 1000 rpm for 10 seconds to precipitate the calluses and whiskers. The supernatant was discarded to obtain the mixture of the calluses and whiskers.

To a tube containing the mixture, 10 μl (10 μg) of the recombinant vector (namely, the recombinant vector pBIH2) and 10 μl (10 μg) of the publicly known recombinant vector pCH having hygromycin resistant gene (Breeding Science: 55, 465-468 (2005)) (hygromycin resistant) were added, and thoroughly shaken to obtain a homogenous mixture.

Subsequently, a tube containing the homogenous mixture was centrifuged at 18000×g for 5 minutes. The centrifuged mixture was again mixed by shaking, and this procedure was repeated 3 times.

A tube containing the callus cells, whiskers and recombinant vector having DNA sequences of the present invention, which were obtained by the above-described method, was installed in the bath of a ultrasonic generator so that it was sufficiently immersed. Ultrasonic waves were irradiated at a frequency of 40 kHz in an intensity of 0.25 w/cm² for 1 minute. After irradiation, the mixture was maintained at 4° C. for 10 minutes. The mixture treated with ultrasonic waves in the above-described method was rinsed with the MS liquid medium to obtain a target transformed callus having the introduced recombinant vector pBIH2.

The transformed callus obtained by introducing the recombinant vector by the above-described method was put in a 3.5 cm petri dish. Furthermore, 3 ml of a liquid medium, which was prepared by adding 30 g/l of sucrose, 500 mg/l of casamino acid, and 6.6 mg/l of dicamba and 0.5 mg/l of benzyladenine as plant hormones, respectively, were added to the inorganic component compositions of MS medium, and adjusting the pH to 5.8. Subsequently, the callus cell was cultured at 28° C. on a rotary shaker (50 rpm) installed in the dark.

After 6 days in culture, the callus cells, to which gene introduction procedure was performed, were uniformly spread on a medium prepared by adding 30 g/l of sucrose, 500 mg/l of casamino acid, and 6.6 mg/l of dicamba and 0.5 mg/l of benzyladenine as plant hormones, respectively, to the inorganic component compositions of publicly known MS medium, adjusting the pH to 5.8, and adding 3 g/l of gel lyte and 100 mg/l of hygromycin as a selection agent. The cell on the medium was cultured at 28° C. in the dark.

After 30 days, the transformed calluses that were healthily growing on the medium containing hygromycin were selected.

(9) Regeneration of a Whole Plant from Transformed Bentgrass Callus Cell

The hygromycin resistant transformed culture cell obtained by the above-described method was transplanted to a medium prepared by adding 30 g/l of sucrose, 500 mg/l of casamino acid and 1 mg/l of benzyladenine as a plant hormone, respectively, to the inorganic component compositions of the MS medium, adjusting the pH to 5.8, and adding 3 g/l of gel lyte. The transformed cultured cell was cultured at 28° C. while irradiating a light at 2000 lux for 16 hours per day. A regenerated plant (budlet) formed after 30 days was transplanted into a test tube containing a medium prepared by adding 30 g/l of sucrose to the inorganic component compositions of MS medium, adjusting the pH to 5.8, and adding 3 g/l of gel lyte (40 mm in diameter, 130 mm in length). The transplanted budlet was cultured for 10 days to obtain a transformed bentgrass whole plant. A total of 2 transformed plants (Strains Nos. BPR1 and BPR2) were obtained from 3 ml of the bentgrass calluses by the above-described method.

(10) Analysis of Introduced Gene

The whole DNAs were extracted from 10 mg of leaf discs of the 2 transformed bentgrass plants obtained in the above (9) in accordance with the publicly known CTAB method. These were checked for the introduced gene by the PCR method. The following primers prepared by chemical synthesis were used in combination as the primers.

(a) Primer No. 1 (represented by SEQ ID NO: 3)
5′-GTGTGATCAGTAGGAAGTTG-3′
(b) Primer No. 2 (represented by SEQ ID NO: 4)
5′-ACCTCAAACACAAATCACTC-3′

One μl of DNA solution of each of the transformed rice strains extracted by the above-described method was used as the template. The amplification reaction solution to be used in the reaction was prepared by a PCR kit (Takara Shuzo Co., Ltd., LA PCR Kit Ver 2.1).

The above-described amplification reaction of DNAs by the PCR method was performed by repeating 35 cycles of 3 reaction procedures, each cycle consisting of denaturation at 94° C. for 30 seconds, annealing at 55° C. for 1 minute and extension at 72° C. for 30 seconds, by using the PCR reaction machine (ASTEK Co., Ltd., Program Temp Control System PC-700).

After PCR, a part of the reaction solution was subjected to electrophoresis in a 1.2% agarose gel to compare the presence and size of the bands. As a result, transformation of the RSI1 gene in the plant of Strain No. BPR1 was confirmed. In Strain No. BPR2, the RSI1 gene was not detected.

(11) Evaluation of Growth of Transformed Bentgrass

The transformed bentgrass plant BPR1, in which the introduction of RSI1 gene was confirmed, and the plant of BPR2, in which the introduction of RSI1 gene was not confirmed, both obtained in the above (9), were transplanted to test tubes containing a medium prepared by adding 30 g/l of sucrose to the inorganic component compositions of the MS medium, adjusting the pH to 5.8, and adding 3 g/l of gel lyte (40 mm in diameter, 130 mm in length). Three plants of each strain were cultured at 28° C. while irradiating a light at 2000 lux for 16 hours per day. Thirty days after the start of culturing, the plant heights and maximum root lengths were measured, and the averages were calculated. The results are shown in Table 4. The plant height and maximum root length of the plant of Strain No. BPR1, in which the introduction of RSI1 gene was confirmed, were significantly larger, compared with those of the plant of BPR2 not having the introduced RSI1 gene, indicating that growth increased.

TABLE 4
Results of evaluation of growth of transformed bentgrass

| | Presence of | Plant height | Maximum root |
|---|---|---|---|
| Strain No. | RSI1 gene RSI1 | (mm) | length (mm) |
| BPR1 | Present | 180 | 44 |
| (Experimental | | | |
| section) | | | |
| BPR2 | Absent | 139 | 17 |
| (Control section) | | | |

The results in Table 4 show that BPR1 having the introduced RSI1 gene clearly exceeded BPR2 not having the introduced RSI1 both in plant height and in root length.

(12) Evaluation of Salt Tolerance of Transformed Bentgrass

The transformed bentgrass plant BPR1 into which the introduction of RSI1 gene was confirmed, and the plant of BPR2 into which the introduction of the RSI1 gene was not confirmed, both obtained in the above (9), were transplanted into test tubes containing a medium prepared by adding 30 g/l of sucrose to 1/10 concentration of the inorganic component compositions of MS medium, adjusting the pH to 5.8, and adding 8 g/l of agar, and then NaCl was added at concentrations of 0, 50, 100, 200 and 300 mM thereto (40 mm in diameter, 130 mm in length). Five plants of each strain were cultured at 28° C. while irradiating a light at 2000 lux for 16 hours per day. Thirty days after the start of culturing, the plant heights and maximum root lengths were measured, and the averages were calculated. The plant height and maximum root length at a NaCl concentration of 0 mM were set to 100%. Relative ratios of the plant height and maximum root length at each NaCl concentration were calculated to obtain the degree of plant height growth and maximum root length growth (%). The results are shown in Table 5. The growth of the plant of Strain No. BPR1, in which the introduction of the RSI1 gene was confirmed, was inhibited, as the results indicated that both the plant height and maximum root length were 98% at a NaCl concentration of 50 mM, indicating that they were mostly not suppressed, approximately 70% at 100 mM, and 30% or less at 200 mM or more. However, the degree of suppression of the plant height and maximum root length associated with the increase in NaCl concentration was smaller, compared with those of the plant of BPR2 not having the introduced RSI1 gene, indicating that salt tolerance is enhanced.

TABLE 5
Results of salt tolerance evaluation of transformed bentgrass

| | | | Degree | Degree of |
|---|---|---|---|---|
| | | | of plant | maximum |
| | Presence | NaCl | height | root |
| | of RSI1 | concentration | growth | length |
| Strain No. | gene | (mM) | (%) | growth (%) |
| BPR1 | Present | 0 | 100 | 100 |
| (Experimental | | 50 | 98 | 98 |
| section) | | 100 | 73 | 69 |
| | | 200 | 34 | 7 |
| | | 300 | 7 | 0 |
| BPR2 | Absent | 0 | 100 | 100 |
| (Control | | 50 | 75 | 68 |
| section) | | 100 | 50 | 46 |
| | | 200 | 13 | 1 |
| | | 300 | 6 | 0 |

### INDUSTRIAL APPLICABILITY

According to the present invention, there is provided an environmental stress tolerant plant in the Poaceae having enhanced resistance to environmental stresses such as salt tolerance and drought tolerance, increased growth, and which can be stably cultivated in areas exposed to various environmental stresses.

1. A method of producing a plant having increased growth, comprising steps of:
introducing DNA into a plant cell to produce a transformed plant cell, wherein the DNA is selected from the group consisting of:
(a) DNA coding for a protein comprising the amino acid sequence represented by SEQ ID NO: 2;
(b) DNA comprising the coding region of the nucleotide sequence represented by SEQ ID NO: 1;
(c) DNA coding for a protein having an amino acid sequence represented by SEQ ID NO: 2 in which one or a few amino acids are substituted, deleted, inserted, and/or added, and having a growth promoting effect;
(d) DNA hybridizing with DNA comprising the nucleotide sequence represented by SEQ ID NO: 1 under stringent conditions and coding for a protein which has a growth promoting effect; and
(e) DNA coding for a protein having an amino acid sequence having 90% or more identity with the amino acid sequence represented by SEQ ID NO: 2 and having a growth promoting effect; and
regenerating a transformed plant from the transformed plant cell.

2. A method of producing a plant having improved heat tolerance, comprising steps of:
introducing DNA into a plant cell to produce a transformed plant cell, wherein the DNA is selected from the group consisting of:
(a) DNA coding for a protein comprising the amino acid sequence represented by SEQ ID NO: 2;
(b) DNA comprising the coding region of the nucleotide sequence represented by SEQ ID NO: 1;
(c) DNA coding for a protein having an amino acid sequence represented by SEQ ID NO: 2 in which one or a few amino acids are substituted, deleted, inserted, and/or added, and being involved in heat tolerance;
(d) DNA hybridizing with DNA comprising the nucleotide sequence represented by SEQ ID NO: 1 under stringent conditions and coding for a protein which is involved in heat tolerance; and
(e) DNA coding for a protein having an amino acid sequence having 90% or more identity with the amino acid sequence represented by SEQ ID NO: 2 and being involved in heat tolerance; and
regenerating a transformed plant from the transformed plant cell.

3. A method of producing a plant having improved heat tolerance and increased growth, comprising steps of:
introducing DNA into a plant cell to produce a transformed plant cell, wherein the DNA is selected from the group consisting of:
(a) DNA coding for a protein comprising the amino acid sequence represented by SEQ ID NO: 2;
(b) DNA comprising the coding region of the nucleotide sequence represented by SEQ ID NO: 1;
(c) DNA coding for a protein having an amino acid sequence represented by SEQ ID NO: 2 in which one or a few amino acids are substituted, deleted, inserted, and/or added, and being involved in a growth promoting effect and heat tolerance;
(d) DNA hybridizing with DNA comprising the nucleotide sequence represented by SEQ ID NO: 1 under stringent conditions and coding for a protein which is involved in a growth promoting effect and heat tolerance; and
(e) DNA coding for a protein having an amino acid sequence having 90% or more identity with the amino acid sequence represented by SEQ ID NO: 2 and being involved in a growth promoting effect; and
regenerating a transformed plant from the transformed plant cell.

4. A method of producing a plant having drought tolerance, comprising steps of:
introducing DNA into a plant cell to produce a transformed plant cell, wherein the DNA is selected from the group consisting of:
(a) DNA coding for a protein comprising the amino acid sequence represented by SEQ ID NO: 2;
(b) DNA comprising the coding region of the nucleotide sequence represented by SEQ ID NO: 1;
(c) DNA coding for a protein having an amino acid sequence represented by SEQ ID NO: 2 in which one or a few amino acids are substituted, deleted, inserted, and/or added, and being involved in drought tolerance;
(d) DNA hybridizing with DNA comprising the nucleotide sequence represented by SEQ ID NO: 1 under stringent conditions and coding for a protein which is involved in drought tolerance; and
(e) DNA coding for a protein having an amino acid sequence having 90% or more identity with the amino acid sequence represented by SEQ ID NO: 2 and being involved in drought tolerance; and
regenerating a transformed plant from the transformed plant cell.

5. A method of producing a plant having salt tolerance, comprising steps of:
introducing DNA into a plant cell to produce a transformed cell, wherein the DNA is selected from the group consisting of:
(a) DNA coding for a protein comprising the amino acid sequence represented by SEQ ID NO: 2;
(b) DNA comprising the coding region of the nucleotide sequence represented by SEQ ID NO: 1;
(c) DNA coding for a protein having an amino acid sequence represented by SEQ ID NO: 2 in which one or a few amino acids are substituted, deleted, inserted, and/or added, and being involved in salt tolerance;
(d) DNA hybridizing with DNA comprising the nucleotide sequence represented by SEQ ID NO: 1 under stringent conditions and coding for a protein which is involved in salt tolerance; and
(e) DNA coding for a protein having an amino acid sequence 90% or more identity with the amino acid sequence represented by SEQ ID NO: 2 and being involved in salt tolerance; and
regenerating a transformed plant from the transformed plant cell.

6. The method according to claim 1, further comprising a step of allowing the plant to undergo sexual reproduction or asexual reproduction, thereby producing a progeny plant.

7. The method according to claim 6, wherein the plant is a monocotyledonous plant.

8. The method according to claim 7, wherein the plant is a rice plant or a bentgrass plant.

9. A plant obtained by the method according to claim 1.

10. The method according to claim 1, wherein the plant is a monocotyledonous plant.

11. The method according to claim 10, wherein the plant is a rice plant or a bentgrass plant.

12. The method according to claim 2, further comprising a step of allowing the plant to undergo sexual reproduction or asexual reproduction, thereby producing a progeny plant.

13. The method according to claim 12, wherein the plant is a monocotyledonous plant.

14. The method according to claim 13, wherein the plant is a rice plant or a bentgrass plant.

15. The method according to claim 2, wherein the plant is a monocotyledonous plant.

16. The method according to claim 15, wherein the plant is a rice plant or a bentgrass plant.

17. A plant obtained by the method according to claim 2.

18. The method according to claim 3, further comprising a step of allowing the plant to undergo sexual reproduction or asexual reproduction, thereby producing a progeny plant.

19. The method according to claim 18, wherein the plant is a monocotyledonous plant.

20. The method according to claim 19, wherein the plant is a rice plant or a bentgrass plant.

21. The method according to claim 3, wherein the plant is a monocotyledonous plant.

22. The method according to claim 21, wherein the plant is a rice plant or a bentgrass plant.

23. A plant obtained by the method according to claim 3.

24. The method according to claim 4, further comprising a step of allowing the plant to undergo sexual reproduction or asexual reproduction, thereby producing a progeny plant.

25. The method according to claim 24, wherein the plant is a monocotyledonous plant.

26. The method according to claim 25, wherein the plant is a rice plant or a bentgrass plant.

27. The method according to claim 4, wherein the plant is a monocotyledonous plant.

28. The method according to claim 27, wherein the plant is a rice plant or a bentgrass plant.

29. A plant obtained by the method according to claim 4.

30. The method according to claim 5, further comprising a step of allowing the plant to undergo sexual reproduction or asexual reproduction, thereby producing a progeny plant.

31. The method according to claim 30, wherein the plant is a monocotyledonous plant.

32. The method according to claim 31, wherein the plant is a rice plant or a bentgrass plant.

33. The method according to claim 5, wherein the plant is a monocotyledonous plant.

34. The method according to claim 33, wherein the plant is a rice plant or a bentgrass plant.

35. A plant obtained by the method according to claim 5.
